(19) United States
(12) Patent Application Publication
(10) Pub. No.: US 2025/0235276 A1
(43) Pub. Date: Jul. 24, 2025

(54) MEDICAL INTERVENTION ASSISTANCE ROBOT

(71) Applicant: Siemens Healthineers AG, Forchheim (DE)

(72) Inventors: Olivia NOACK, Bayreuth (DE); Claudia LINDNER, Nuernberg (DE)

(73) Assignee: Siemens Healthineers AG, Forchheim (DE)

(21) Appl. No.: 19/032,331

(22) Filed: Jan. 20, 2025

(30) Foreign Application Priority Data
Jan. 23, 2024 (EP) 24153399.1

(51) Int. Cl. A61B 34/37; A61B 34/00; A61B 50/13; A61B 50/15; A61B 50/30; A61L 2/10; A61L 2/18; A61L 2/22; A61L 101/34

(52) U.S. Cl. CPC A61B 34/37; A61B 34/25; A61B 50/13; A61B 50/15; A61B 50/30; A61L 2/10; A61L 2/18; A61L 2/22; A61L 2101/34; A61L 2202/11; A61L 2202/122; A61L 2202/16; A61L 2202/24

(57) ABSTRACT

A medical intervention assistance robot is described. The medical intervention assistance robot comprises a first operating arm configured to handle a non-sterile object, a second operating arm configured to handle a sterile object and a support unit configured to support the first operating arm and the second operating arm. The medical intervention assistance robot also comprises a control unit configured to control a movement of the first operating arm and a movement of the second operating arm. Further, a medical intervention method using a medical intervention assistance robot is described.

### CROSS-REFERENCE TO RELATED APPLICATION(S)

The present application claims priority under 35 U.S.C. § 119 to European Patent Application No. 24153399.1, filed Jan. 23, 2024, the entire contents of each of which are incorporated herein by reference.

### FIELD

One or more example embodiments relates to a medical intervention assistance robot. One or more example embodiments also refers to a medical intervention method using a medical intervention assistance robot.

### RELATED ART

In clinical routine, every single procedure needs to be prepared. For interventions, guided by medical imaging, these preparations include for example the step of cleaning and disinfection of the patient table, the step of setting up and cleaning a sterile table, equipping a table with needles, devices, skin disinfection, swabs, scalpels etc. Particularly for CT-guided interventions, the preparations also comprise the steps of setting up intervention hardware accessories, for example a joystick or a tablet holder, orienting dedicated intervention hardware and monitors and setting up sterile covers on gantry, tablet or remote control.

Typically, these preparational tasks are performed by technicians or the doctor themselves and take several minutes. Preparation basically depends on the preferences of the doctor and type of procedure to come next. Overall, the preparation can be summarized as highly repetitive and predictable tasks without direct impact on clinical outcome.

During an ideal procedure, the radiologist is supported by at least one technician. Some of those tasks performed by the technician, are being a third hand and providing flasks, meds, open packages in a way that radiologist can remain sterile.

There are even more tasks, typically performed by a technician. The tasks vary strongly in their complexity level. Typical and simple technician tasks also involve opening and closing doors, get whatever material might be used from for example drawers in the treatment room.

### SUMMARY

So far, preparation is one of the typical technician tasks, respectively also performed by interventional radiologists themselves. However, this approach is not future-proof due to increasing staff shortage in the healthcare sector. It is already observed nowadays that scanners are only used at reduced hours due to personnel lack implying less performed procedures.

It is therefore a task to provide a system for automatically assisting medical interventions of different types, from diagnostics to treatment or surgery.

This task is accomplished by a medical intervention assistance robot according one or more example embodiments and/or by a medical intervention method using a medical intervention assistance robot according one or more example embodiments.

One or more example embodiments are explained below with reference to the figures. The same components are provided with identical reference numbers in the various figures.

The figures are usually not to scale.

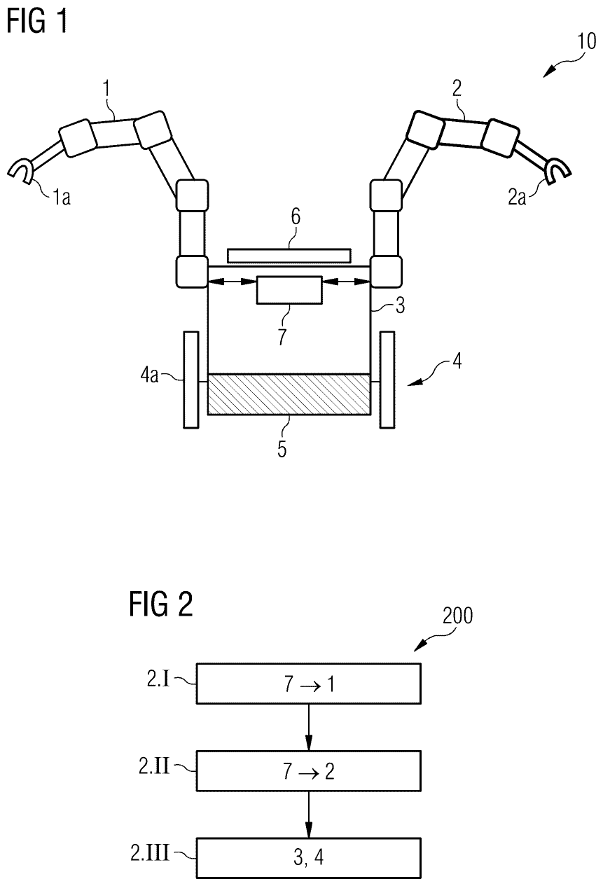

FIG. 1 shows a schematic illustration of a medical intervention assistance robot according to an embodiment of the invention, and

FIG. 2 shows a flow chart, illustrating a medical intervention method using an assistance robot according to an embodiment of the invention.

### DETAILED DESCRIPTION

The medical intervention assistance robot according to one or more example embodiments comprises a first operating arm for handling a non-sterile object, a second operating arm for handling a sterile object and a support unit for supporting the first operating arm and the second operating arm. The expression “sterile” means that the sterile object is germ-free or almost germ-free. Sterilization is normally performed using high temperature media like hot steam of hot air or using UV radiation. Compared to Sterilization, disinfection enables only a reduction of germs or pathogens. Disinfection is preferably performed using chemical disinfection substances. However, it has to be emphasized that the expressions “disinfection” and “sterilization” are widely used as a synonym in the application.

The first operating arm is preferably used, in some example embodiments, for handling non-sterile objects for preparing an intervention or for supporting actions during an intervention or for completing an intervention. Preferably, the first operating arm includes a gripping unit for gripping a non-sterile object. Preferably, the gripping unit is designed with finger-like gripping tools for holding an object and for processing the object in parallel. For example, a package as a special type of an object is held with a first finger-like gripping tool and the package is opened in parallel with another finger-like gripping tool of the same operating arm. Hence, it is preferred that the gripping unit comprises at least two independent finger-like gripping tools or constructs, more preferably even three such finger-like gripping tools or constructs. The gripping unit is preferably fixed to the first operating arm. Alternatively, the gripping unit is arranged to be easily removable from the first operating arm. It is preferred that the first operating arm is designed such that it is cleanable very well and such that it is constructed based on hygienic design principles.

The second operating arm is preferably used, in some example embodiments, for handling sterile objects for preparing an intervention or for supporting actions during an intervention or for completing an intervention. For handling sterile objects, the second operating arm preferably comprises a sterile gripping unit. Preferably, the sterile gripping unit is designed to be easily removable from the second operating arm. The second operating arm preferably comprises a surface material which is easily cleanable and disinfectable or even sterilizable.

Preferably, the first operating arm and the second operating arm comprise actor units for moving them to a predetermined position or pose.

The medical intervention assistance robot according to one or more example embodiments further comprises a control unit for controlling the movement of the first operating arm and the second operating arm. The control unit is preferably designed to transmit control orders to the actor units of the first operating arm and the second operating arm for moving the first operating arm and the second operating arm to a predetermined position or pose.

Preferably, the control unit is designed to control an interplay or an interaction between the first operating arm and the second operating arm. In particular, such an interaction can be advantageously performed by using the sterile second operating arm for holding a sterile package and by using the first non-sterile operating arm for putting a non-sterile object into a package or for removing the non-sterile object from the package. Such, an object, in particular an accessory, can be automatically packed or removed without any need of human support.

Preparation may comprise one of the following types of preparation action:
a preparation of an imaging modality, in case the medical imaging modality is used for guiding an intervention, in particular such a medical imaging modality may comprise a CT scan unit or an MRT scan unit or an angiography scan unit, in particular an angiography CT scan unit, or an ultrasonic scan unit, respectively,
a procedure preparation for preparing the intervention procedure,
in particular cleaning and disinfection the patient table,
positioning the patient at the correct position on the patient table,
finalizing the intervention set-up,
supporting the patient with sterile draping,
supporting the interventionalist with dressing sterile.

Typical supporting actions during an intervention comprise:
providing an autonomous sterile table,
assisting the interventionalist during the intervention procedure,
non-sterile interaction with environment.

Typical actions for completing an intervention comprise:
clearing the scan unit of an imaging modality, in case such a scan unit, in particular one of the above-mentioned special types, is used for guiding intervention,
cleaning the scan unit, in case a scan unit is used for guiding intervention,
unmounting equipment and bring back the equipment to stock,
emptying a sterile table,
cleaning a patient table,
cleaning a gantry,
cleaning a sterile table.

For example, for preparation, the first operating arm may be used for some henchman tasks like cleaning a table, in particular a patient table, or a gantry using a spray and/or a wipe. The first operating arm may also be used for fetching and/or setting up required need for interventions regardless of modality or in case of the use of a CT-system, accessories. Such need for interventions may comprise incidentals like swabs, needles, medications, disinfectants, in particular povidone-iodine, plasters or scalpels. The first operating arm may also be used for aligning hardware components or for passing or holding local anesthesia flasks or other types of med flasks or for opening packages.

Further, the first operating arm may be used during the intervention procedure for holding syringes, flasks, for unpacking disposables and for mixing medication by shaking.

Further, the first operating arm can be used for opening or closing a door or for changing light settings or for operating non-covered components, for example an ablation generator. Furthermore, the first operating arm is preferably used for removing covers or for unmounting accessories or for cleaning them in some example embodiments.

Preferably, the first operating arm is used for getting positioning aids for positioning a patient to be examined in some example embodiments. These are differently shaped cushion-like cushions so that the patient can be positioned in a stable position. These are usually stored on shelves in the room or rarely in the neighboring room (if not needed).

In some example embodiments, the second operating arm is preferably used as a third hand for sterile operations. In particular, the third operating arm is used for equipping a sterile table, for assistance during procedure or for sterile covering the patient or an object. The second operating arm is also used for unpacking a coat or gloves or for being a third hand to hold the one-way cloths in proper position for the doctor.

Further, the second operating arm is preferably used for setting-up or for disinfecting a sterile table and/or for equipping the sterile table with needles or devices or skin disinfectant or swabs or scalpels.

Preferably, the control unit is also used for synchronized controlling the movement of the first operating arm and the second operating arm such that the first operating arm and the second operating arm work together with each other. For example, the first operating arm holds a disinfection flask for disinfection of a surface or for disinfecting a human injection site in particular using iodine. The second operating arm performs all actions comprising the contact with objects or surfaces which may be touched by the sterile gloves of the doctor or interventionalist after a contact with the second operating arm. Interplay of both robotic arms is preferably needed for sterile packing of components, for unpacking and providing flasks and for supporting interventional radiologist in dressing sterile.

Advantageously, the availability of an intervention assistance robot is higher than the availability of a human technician. Further, the intervention assistance robot reduces the regular costs for salary of employees. Generally, the number of the needed persons for an intervention is reduced due to the automatic assistance. The combination of a first operating arm for handling a non-sterile object and a second arm for handling a sterile object enables to automatically carry out transport actions and unpacking and packing actions concerning sterile objects packed in non-sterile covers. Advantageously, also an interplay of both robotic arms is enabled, in particular for sterile packing of components like a tablet or a remote control and/or for unpacking and/or providing objects like flasks.

According to the method for using a medical intervention assistance robot, which is preferably a medical intervention assistance robot according to one or more example embodiments, according to one or more example embodiments, a first operating arm for handling a non-sterile object is used and a second operating arm for handling a sterile object is used, wherein the first operating arm and the second operating arm are supported using a support unit. Advantageously, non-sterile objects can be handled without contaminating the second arm dedicated to handling sterile objects.

Some units or modules of the medical intervention assistance robot mentioned above can be completely or partially realized as software modules running on a processor of a respective computing system, e.g. of the control unit of the medical intervention assistance robot. A realization largely in the form of software modules can have the advantage that applications already installed on an existing computing system can be updated, with relatively little effort, to install and run these units of the present application. The is also achieved by a computer program product with a computer program that is directly loadable into the memory of a computing system, in particular a control unit, and which comprises program units to perform the step of controlling the movement of the first operating arm and the second operating arm of the inventive method for using the first operating arm and the second operating arm, when the program is executed by the computing system. In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

The dependent claims and the following description each contain particularly advantageous embodiments and developments of one or more example embodiments. In particular, the claims of one claim category can also be developed analogously to the dependent claims of another claim category. In addition, within the scope of the invention, the various features of different exemplary embodiments and claims can also be combined to form new exemplary embodiments.

Preferably, the intervention for which the medical intervention assistance robot is used, comprises an image-guided intervention. For imaging, a medical imaging system may be used, in particular one of the following types: a computer tomography system, a magnetic resonance imaging system, an ultrasound system, an X-ray system, in particular a planar X-ray system or an angiography system or a C-arm system or an angiography CT system. The intervention assistance robot can alternatively be used for blind interventions, for which sterility needs to be maintained and sterile equipment must be prepared. Advantageously, the interventionalist is enabled to perform interventions autonomously in an efficient way.

In a variant of the medical intervention assistance robot according to one or more example embodiments, the medical intervention assistance robot according to one or more example embodiments comprises a mobility unit for mobilizing the robot. Advantageously, the mobility unit enables the robot to be mobile such that the robot can trans-port required objects over longer distances and is able to save the doctor from having to walk around the treatment room or outside the treatment room himself. The mobility unit enables re-stocking, fetching and/or storing accessories etc.

In some example embodiments the mobility unit includes rolling elements for moving in all directions. Rolling elements, particularly wheels, enable to move a robot with low energy expense and very smooth without any shock such that equipment being transported on the robot does not fall down from the robot.

Particularly preferred, the rolling elements comprise one of the following types:
a Mecanum wheel,
an all-side wheel.

Advantageously, the mentioned types of wheels enable to change the moving direction abruptly and with limited space for the change of the moving direction.

The intervention assistance robot according to one or more example embodiments includes a sterilization chamber. Advantageously, a sterile condition of the second operating arm can be assured in every situation.

Further, the medical intervention assistance robot according to one or more example embodiments includes a sterile table, preferably on the top of the support unit of the medical intervention assistance robot. Preferably, the sterile table is designed to be attached to a surgical rail, in particular at a scanner, so that the medical intervention assistance robot can move freely during interventions.

Most patient tables have mounted rails on the sides, these are typically made of stainless steel and are used to attach things to them. There are not any number of different variants of these rails, but rather a standard has emerged, which is referred to as “surgical rail”.

In some example embodiments, the sterile table comprises as material stainless steel. Advantageously, the table is inert against disinfection fluids.

In some example embodiments, the sterile table or the sterile chamber is arranged to keep an object sterile based on one of the following methods:
using a disinfection spray,
using a wipe,
radiating with UV light.

Radiating with UV light enables to disinfect or even sterilize an object without contacting with a sterilization or disinfection agent or means. For example, the sterilization or disinfection agent may cause an allergic reaction. Using a wipe enables to mechanically clean an object additionally to a sterilization or disinfection of the object.

For example, the sterilization or disinfection can be performed using the first and second operating arms, wherein the first operating arm holds the disinfection or sterilization spray and the object to be disinfected or sterilized is positioned on the sterile table. In an alternative variant, the object to be disinfected or even to be sterilized is positioned in the sterile chamber and is disinfected or even sterilized therein, preferably using a UV light, in particular UV-C light.

The medical intervention assistance robot according to one or more example embodiments further preferably includes a storage section. Advantageously, objects necessary or useful for an intervention can be stored in the storage section.

Preferably, the first operating arm of the medical intervention assistance robot according to one or more example embodiments is arranged to execute any of the following handling functions:
fetching an object,
mounting an object,
orienting an object,
holding an object.

Advantageously, a doctor tasked with carrying out an intervention, is exonerated from procuring and preparing an object being necessary for the intervention.

In some example embodiments, the first operating arm of the medical intervention assistance robot according to one or more example embodiments is arranged to execute one of the following actions:
cleaning a patient table,
cleaning a gantry,
getting a disposables from the stock,
open or close a door,
passing or holding a local anesthetic or a med flask,
open a package,
interacting with a non-sterile covered part of an imaging system,
interacting with a non-sterile covered machine of a type being different from an imaging system,
supporting the interventionalist to become sterile.

Advantageously, a doctor can concentrate completely on the actual intervention, since the intervention assistance robot takes over all the henchman's work for him.

The second operating arm of the medical intervention assistance robot according to one or more example embodiments is preferably arranged to execute one of the following actions:
covering a gantry,
packing a tablet and/or a remote control in a sterile bag,
supporting with covering a patient.

Packing tablets and/or remote controls in sterile bags is performed by an interaction of the first operating arm and the second operating arm, since the bags are sterile only on the outside and non-sterile on the inner side just as the object inside the bags.

Advantageously the environment of the intervention is kept sterile so that an infection of a patient can be avoided.

The control unit of the medical intervention assistance robot according to one or more example embodiments is preferably arranged to function based on one of the following communication principles:
gesture recognition,
voice recognition,
a touch display interaction function.

Advantageously, the intervention assistance robot is enabled to be controlled by a doctor tasked with an intervention using common means of communication for control such as through a user interface.

Further, the intervention assistance robot optionally comprises technical means for exploring the environment and for enabling communication with a human person. In particular, these technical means include sensor means or communication means. Preferably these technical means comprise at least one of the following types of technical means:
a sensor unit,
a camera,
a display,
speakers.

Advantageously, the sensor unit and the camera can be used for perceiving the surroundings of the intervention assistance robot, in particular for localizing and gripping an object or for driving through the treatment room without any collision.

The display and the speakers can be used for transmitting information by the intervention assistance robot to the intervening doctor or vice versa. For example, the intervention assistance robot is enabled to communicate with the intervening doctor such that the assistance robot provides the doctor with a required object.

In FIG. 1, a schematic illustration of a medical intervention assistance robot 10 according to an embodiment of the invention is depicted. The medical intervention assistance robot 10 comprises a first operating arm 1 for handling a non-sterile object. The first operating arm 1 is illustrated on the left side of FIG. 1. The first operating arm 1 comprises a gripping unit la for gripping non-sterile objects. Further, the medical intervention assistance robot 10 comprises a second operating arm 2 for handling a sterile object. The second operating arm 2 is illustrated on the right side of FIG. 1. The second operating arm 2 comprises a gripping unit 2a for gripping a sterile object. The medical intervention assistance robot 10 also comprises a support unit 3 for supporting the first operating arm 1 and the second operating arm 2. The support unit 3 is illustrated in the center of FIG. 1. Part of the support unit 3 is also a mobility unit 4 including wheels 4a for changing the position of the medical intervention assistance robot 10. On the top of the support unit 3, a sterile table 6 is arranged for transporting sterile objects which can mainly be handled by the second operating arm 2. The support unit 3 also comprises a sterilization chamber 5 for implementing a sterilization process for the sterile table 6 or the sterile part of the robotic arm, i.e. the gripping unit 2a of the second operating arm 2. The sterilization process is realized using disinfection chemicals like ethanol and UV light. Further, the medical intervention assistance robot 10 comprises a control unit 7 for controlling the movement of the first operating arm 1 and the second operating arm 2.

In FIG. 2 a flow chart 200, illustrating a medical intervention method using a medical intervention assistance robot 10 according to an embodiment of the invention is shown.

In step 2.I, a control order is emitted by a control unit 7 of a medical intervention assistance robot 10 for controlling a first operating arm 1 or gripping a package whose outer surface is non-sterile. The package contains a sterile object, for example a scalpel. The first operating arm 1 is also used for opening the package.

In step 2. II, a control order is emitted by the control unit 7 of the medical intervention assistance robot 10 for controlling a second operating arm 2 for handling a sterile object for removing the scalpel from the package. It has to be mentioned that the step 2.I and step 2. II can be carried out at the same time or sequentially.

In step 2.III, the supporting unit 3 of the medical invention assistance robot 10, including a mobility unit 4, is used for transporting objects for a pre- and post-preparation step and for transporting objects for the intervention itself, like a scalpel, which is kept hold of the second operating arm 2, to a doctor standing at an operating table.

In an alternative scenario, the doctor or interventionalist takes everything he can easily handle with one hand from the sterile table 6 of the robot 1. In the alternative scenario, the doctor or interventionalist only changes his position very rarely during the most types of intervention, i.e. driving behind the doctor or interventionalist is not frequently needed in most scenarios.

In these scenarios, the mobile support of the robot relates more to the pre-and post-preparation step or when supplies are needed or interaction with the environment is required.

Further, there is another use case in which the robot could offer added value if it delivers in a sterile manner, possibly with a change of location, namely when doctor progresses the needle partially outside and inside of the bore for a CT-guided needle intervention.

This means that the patient table for the needle advance is not moved out of the gantry, but rather the doctor or interventionalist leans into it. The reasons are either to save time or because live imaging (fluoroscopy) is required.

Finally, it should be pointed out once again that the detailed methods and structures described above are exemplary embodiments and that the basic principle can also be varied in wide areas by the person skilled in the art without leaving the scope of the invention, insofar as it is specified by the claims. For the sake of completeness, it should also be noted that the use of the indefinite articles “a” or “an” does not exclude the fact that the characteristics in question can be present multiple times. Likewise, the term “unit” does not exclude the fact that it consists of several components, which may also be spatially distributed. Further, independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being The software also may be interpreted by, a hardware device. distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax languages from including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A medical intervention assistance robot, comprising:
a first operating arm configured to handle a non-sterile object;
a second operating arm configured to handle a sterile object;
a support unit configured to support the first operating arm and the second operating arm; and
a control unit configured to control a movement of the first operating arm and a movement of the second operating arm.

2. The medical intervention assistance robot of claim 1, further comprising:
a mobility unit configured to mobilize the robot.

3. The medical intervention assistance robot of claim 2, wherein the mobility unit includes rolling elements for moving in different directions.

4. The medical intervention assistance robot of claim 3, wherein the rolling elements comprise one of the following types:
a Mecanum wheel, and
an all-side wheel.

5. The medical intervention assistance robot of claim 1, further comprising:
a sterilization chamber.

6. The medical intervention assistance robot of claim 1, further comprising:
a stainless steel sterile table on a top of the support unit.

7. The medical intervention assistance robot of claim 6, wherein the sterile table is arranged to keep an object sterile based on at least one of the following methods:
using a disinfection spray,
using a wipe, and
radiating with UV light.

8. The medical intervention assistance robot of claim 1, further comprising:
a storage section.

9. The medical intervention assistance robot of claim 1, wherein the first operating arm is configured to execute at least one of:
fetching an object,
mounting an object,
orienting an object, and
holding an object.

10. The medical intervention assistance robot of claim 1, wherein the first operating arm is configured to execute at least one of:
cleaning a patient table,
cleaning a gantry,
getting disposables from a stock,
open or close a door,
passing or holding a local anesthetic or a med flask,
open a package,
interacting with a non-sterile covered part of an imaging system,
interacting with a non-sterile covered machine of a type being different from an imaging system, and
supporting an interventionalist to become sterile.

11. The medical intervention assistance robot of claim 1, wherein the second operating arm is configured to execute at least one of:
covering a gantry,
packing at least one of a tablet or a remote control in a sterile bag, and
supporting covering a patient.

12. The medical intervention assistance robot of claim 1, wherein the control unit is controlled based on one of:
gesture recognition,
voice recognition, and
a touch display interaction function.

13. The medical intervention assistance robot of claim 1, wherein the control unit is configured to control an interaction between the first operating arm and the second operating arm.

14. The medical intervention assistance robot of claim 1, wherein the control unit is configured to synchronize control of the first operating arm and the second operating arm such that the first operating arm and the second operating arm work together with each other.

15. A medical intervention method using a medical intervention assistance robot, the method comprising:
using a first operating arm to handle a non-sterile object;
using a second operating arm to handle a sterile object;
supporting the first operating arm and the second operating arm using a support unit; and
controlling a movement of the first operating arm and a movement of the second operating arm using a control unit.

16. The medical intervention method of claim 15, the method comprising:
controlling an interaction between the first operating arm and the second operating arm.

17. The medical intervention method of claim 15, the method comprising:
synchronized controlling the movement of the first operating arm and the movement of the second operating arm such that the first operating arm and the second operating arm work together with each other.

18. A computer program product comprising instructions which, when executed by a computer, cause the computer to perform the method of claim 15.

19. A non-transitory computer-readable storage medium comprising instructions which, when executed by a computer, cause the computer to perform the method of claim 15.
